(19) United States
(12) Patent Application Publication
(10) Pub. No.: US 2013/0144253 A1
(43) Pub. Date: Jun. 6, 2013

(54) CATHETER ELECTRICAL CONNECTOR ASSEMBLY

(71) Applicants: Garrett Ryan, Surry Hills (AU); Samar Sourour, Lidcombe (AU)

(72) Inventors: Garrett Ryan, Surry Hills (AU); Samar Sourour, Lidcombe (AU)

(73) Assignee: CATHRX LTD, NEW SOUTH WALES, AU (AU)

(21) Appl. No.: 13/816,715

(22) PCT Filed: Aug. 12, 2011
(86) PCT No.: PCT/AU2011/001020

§ 371 Date: Feb. 12, 2013

Related U.S. Application Data
(60) Provisional application No. 61/373,719, filed on Aug. 13, 2010.

(51) Int. Cl. H01R 13/46; A61M 25/01

(52) U.S. Cl. CPC H01R 13/46; A61M 25/0105
USPC 604/500; 439/577; 604/264

(57) ABSTRACT

A catheter includes a catheter sheath, and a handle. The handle includes an electrical connector its proximal end for connecting the catheter to standard electrical devices such as a typical monitoring device. The catheter handle houses an electrical connector assembly. The electrical connector assembly includes a first connector member to which electrical conductors of a catheter sheath are connected and a second connector member, which is further connected to the connector integrated at the proximal end of the catheter handle. The first connector member is received in a socket of the second connector member in a clip-in manner.

### CROSS-REFERENCE TO RELATED APPLICATIONS

This application is a national phase entry under 35 U.S.C. §371 of International Patent Application PCT/AU2011/001020, filed Aug. 12, 2011, designating the United States of America and published in English as International Patent Publication WO 2012/019231 A1 on Feb. 16, 2012, which claims the benefit under Article 8 of the Patent Cooperation Treaty and under 35 U.S.C. §119(e) to U.S. Provisional Patent Application Ser. No. 61/373,719, filed Aug. 13, 2010, the disclosure of each of which is hereby incorporated herein by this reference in its entirety.

### TECHNICAL FIELD

This disclosure relates, generally, to the field of catheters and, more particularly, to a catheter electrical connector assembly and to a catheter assembly including the electrical connector assembly.

### BACKGROUND

Any discussion of the prior art throughout the specification should in no way be considered as an admission that such prior art is widely known or forms part of common general knowledge in the field.

Catheters are becoming increasingly more complex and, as a result, more expensive. Generally, catheters are used only once and are then disposed of. This results in significant expense for the institution using the catheters. Ultimately, these expenses are passed on to the patient. This results in medical costs increasing for both the patient and medical funds, be they private or government funds.

It would be beneficial to be able to reprocess catheters. A problem with reprocessing of catheters is that the electrical connections made between the catheter sheath and the handle are secured connections, which are difficult to disconnect. The electrical connector itself at a proximal end of the handle is also an expensive item. Thus, ideally, it would be beneficial to be able to re-use the electrical connector.

To reprocess the catheters, non-skilled labor is often used. Such non-skilled labor may not have the necessary abilities to be able to disconnect the catheter sheath from the electrical connector of the handle in an efficient manner. Also, with a secure connection between the conductors of the catheter sheath and the connector, special tools may be required that are easy to misplace and, without the tools, it is not possible to effect disconnection of the catheter sheath from the handle in a re-usable manner.

### BRIEF SUMMARY

It is an object of the present invention to overcome or ameliorate at least one of the disadvantages of the prior art, or to provide a useful alternative.

In a first aspect, there is provided a catheter electrical connector assembly that includes:
a first connector member to which conductors of a catheter sheath are connectable; and
a second connector member to which conductors of an electrical connector of a catheter handle are connectable, the first connector member and the second connector member having complementary engaging formations for effecting disconnection between the first connector member and the second connector member, the first and second connector members further being of a compact construction to facilitate mounting in the catheter handle.

“Effecting disconnection” is to be understood to mean that the two connector members may be connected and disconnected easily, e.g., in a clip-in manner, from one another so that no tools are required to disconnect the connector members from each other. In addition, “compact construction” is to be understood to mean that, when the connectors, connected to each other, are mounted in the handle, there is still space in the handle for other items to pass the connected connectors. The other items may be a stylet, an irrigation conduit, or the like.

The first connector member may have a substantially planar configuration having a surface carrying conductive pads, the conductors of the catheter sheath being electrically connectable to the conductive pads.

The second connector member may comprise a receptacle defining a socket housing terminals, the first connector member being receivable in the socket, preferably slidably, with each pad of the first connector member being associated with one of the terminals of the receptacle.

The first connector member may have a configuration similar to a SIM card, compact memory card, or the like. Thus, the receptacle of the second connector member may be shaped to accommodate the card-shaped first connector member. If necessary or desired, the receptacle may comprise a retaining element, such as a clip, for retaining the first connector member in the receptacle of the second connector member with the retaining member being manipulated to effect release of the first connector member from the second connector member.

The terminals of the receptacle may be connectable via a cable arrangement to the electrical connector.

In an embodiment, the second connector member may comprise a printed circuit board on which the receptacle is mounted, the cable arrangement extending from the printed circuit board. In another embodiment, the cable arrangement may comprise a flexible printed circuit board on which the receptacle is mounted, the flexible printed circuit board being connectable to the electrical connector.

In a second aspect, there is provided a catheter electrical connector assembly that includes:
a first, card-shaped connector member to which conductors of a catheter sheath are connectable; and
a second connector member to which conductors of an electrical connector of a catheter handle are connectable, the second connector member defining a socket formation in which the first connector member is removably receivable.

In a third aspect, there is provided a catheter assembly that includes:
a handle having a proximal end and a distal end and a passage extending between the proximal end and the distal end;
an electrical connector arranged at the proximal end of the handle with the electrical connector being in communication with the passage of the handle;
a catheter sheath extending from the distal end of the handle, the catheter sheath carrying at least one electrode at a distal region of the catheter sheath with conductors for the catheter sheath extending along the catheter sheath proximally to extend beyond a proximal end of the catheter sheath; and
an electrical connector assembly, as described above, received in the passage of the handle with the conductors of the catheter sheath connected to the first connector member and the second connector member being electrically connected to the electrical connector.

The catheter sheath may define at least one lumen and the conductors extend through the lumen.

The catheter assembly may include a protective element that is received over those portions of the conductors extending beyond the proximal end of the catheter sheath.

The protective element may be a sleeve acting as a strain relief. The sleeve may be of a heat-shrink material.

### BRIEF DESCRIPTION OF THE DRAWINGS

Preferred embodiments of the invention will now be described, by way of example only, with reference to the accompanying drawings in which:

FIG. 1 shows a perspective view of an embodiment of a catheter assembly;

FIG. 2 shows a schematic, cross-sectional side view of a first embodiment of a catheter electrical connector assembly;

FIG. 3 shows a schematic, cross-sectional side view of a second embodiment of a catheter electrical connector assembly;

FIG. 4 shows a schematic, perspective view of a first connector member of the assembly of FIG. 2 or FIG. 3; and

FIG. 5 shows a plan view of an example of a second connector member of the assembly of FIG. 2 or FIG. 3.

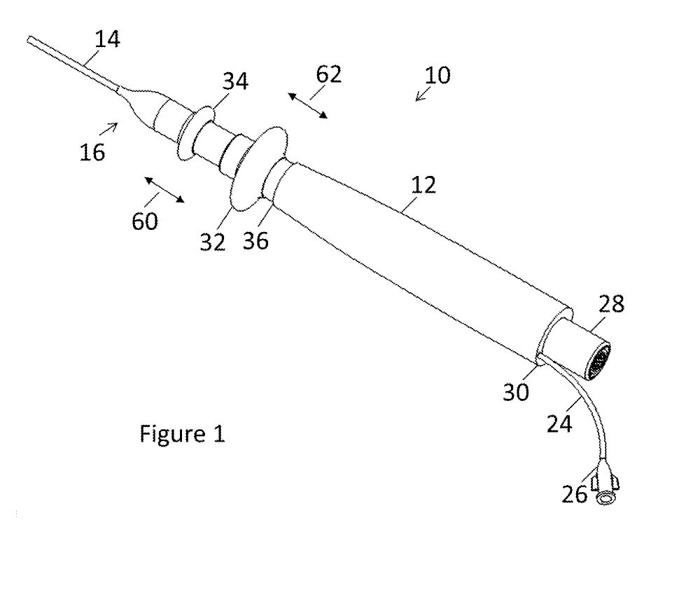

### DETAILED DESCRIPTION

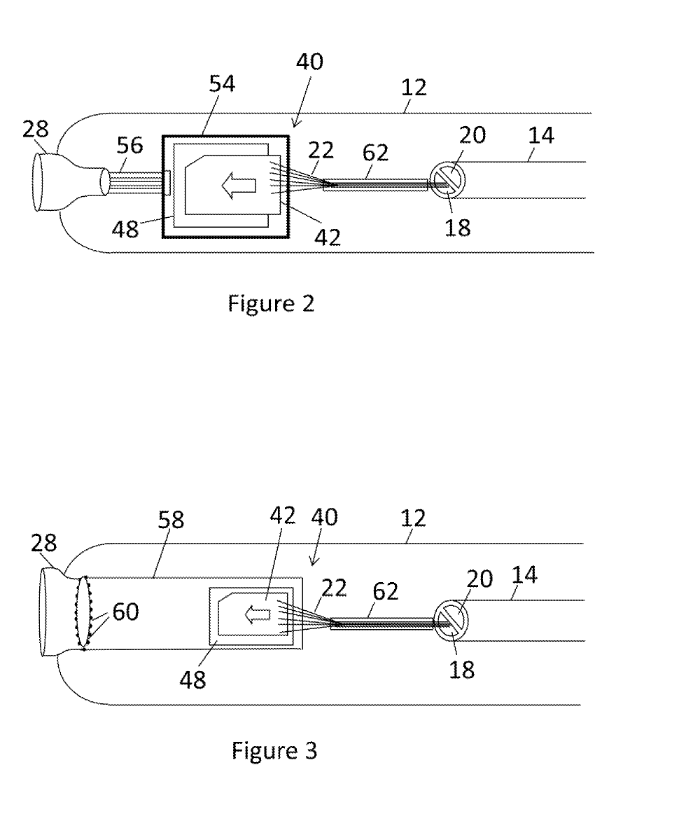

In the drawings, reference numeral 10 generally designates an embodiment of a catheter assembly. The catheter assembly includes a handle body 12. A catheter sheath 14 extends from a distal end 16 of the handle arrangement. The catheter sheath 14 defines a plurality of lumens, illustrated schematically at 18 and 20 in FIGS. 2 and 3. The lumen 18 is a conductor lumen and has a plurality of conductors 22 received therein. The lumen 20 is a deflection stylet lumen for receiving a deflection stylet (not shown). While not illustrated, the catheter sheath 14 includes a further lumen, being an irrigation lumen. The irrigation lumen of the catheter sheath 14 has a proximal end in communication with a fluid supply conduit 24 having a Luer connector 26 arranged at the proximal end of the conduit 24.

An electrical connector 28 is arranged at a proximal end 30 of the handle body 12. The catheter assembly 10 further includes deflection and size selection units 32 and 34, respectively, arranged at a distal end 36 of the handle body 12.

Referring now to FIGS. 2 and 3 of the drawings, two embodiments of an electrical connector assembly 40 of the catheter assembly 10 are described in greater detail.

The purpose of the connector assembly 40 is to connect the electrical conductors that transmit electrical signals from or to the electrodes located at the distal end of the catheter sheath 14 to a patient monitor or similar via the connector unit 28. The connector assembly 40 comprises a first connector member 42 to which the conductors 22 of the catheter sheath 14 are connected. In the embodiments illustrated, the connector member 42 has a substantially planar configuration. Preferably, the connector member 42 has a configuration similar to a SIM card, a micro SD card, a compact memory card, or the like.

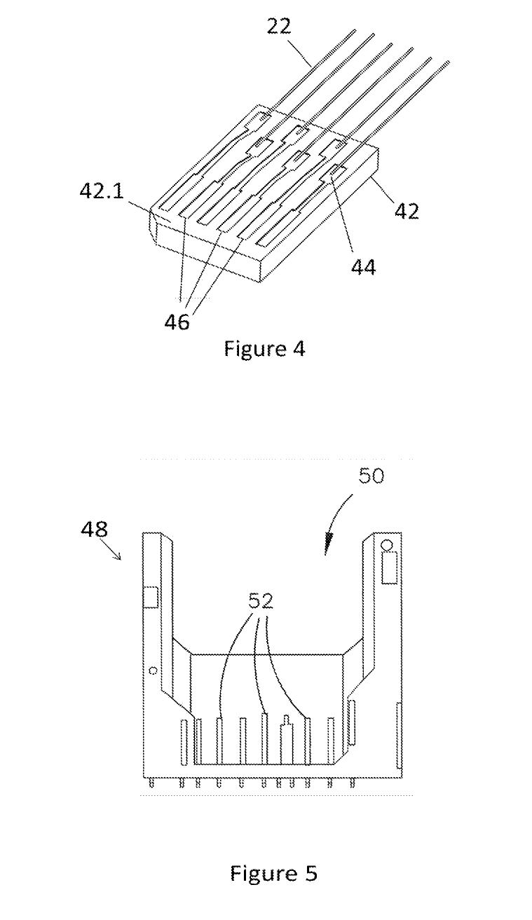

The first connector member 42 defines a surface 42.1 (see FIG. 4) on which the conductors 22 of the catheter sheath 14 terminate in terminations 44. These terminations 44 are, in turn, connected to electrically conductive pads 46 (FIG. 4) on the surface 42.1. The conductors 22 are permanently connected to the first connector member 42 by welding or by another suitable method so that the signals transmit from the electrical conductors 22 to the conductive pads 46 of the first connector member 42.

The connector assembly 40 further includes a second connector member 48 arranged in the handle body 12. The second connector member 48 receives the first connector member 42 therein, preferably, in a slidable manner or in a clip-in manner. The second connector member 48 is, therefore, in the form of a receptacle that defines a socket 50 (FIG. 5) in which the planar first connector member 42 is received. The second connector member 48 has a plurality of terminals 52 (FIG. 5) within the socket 50, the terminals 52 mating with the pads 46 of the first connector member 42 when the first connector member 42 is received in the socket 50 of the second connector member 48.

In the embodiment of the electrical connector assembly 40 illustrated in FIG. 2, the second connector member 48 is surface mounted on a printed circuit board 54. The printed circuit board 54 is, in turn, connected via a plurality of connecting wires 56, in the form of a ribbon cable or soldered wires, to the connector 28.

It will, therefore, be appreciated that when it is desired to replace the catheter sheath 14, the first connector member 42 is disconnected from the second connector member 48 by sliding it out of the second connector member 48 or unclipping it from the second connector member 48. Thus, no special tools are required to disconnect the first connector member 42 from the second connector member 48. The sub-assembly comprising the second connector member 48, the circuit board 54 and the connector 28 remain in the handle body 12 for reprocessing and re-use.

In the embodiment of the electrical connector assembly shown in FIG. 3, the second connector member 48 is mounted on a flexible printed circuit board 58, which, in turn, is directly connected to pins 60 of the electrical connector 28.

In this embodiment, once again, to remove the catheter sheath 14 from the catheter assembly 10, the first connector member 42 is disconnected from the second connector member 48 by sliding the first connector member 42 out of the second connector member 48 or unclipping the first connector member 42 from the second connector member 48. Thus, once again, no special tools are required to disconnect the first connector member 42 from the second connector member 48.

Likewise, no special tools are required, in either embodiment, to re-connect the first connector member 42 and the second connector member 48.

In both embodiments, to protect those portions of the conductors 22 of the catheter sheath 14 projecting proximally from the proximal end of the catheter sheath 14, a protective heat-shrink sleeve 62 is applied to the conductors 22. This also serves to retain the conductors 22 in a neat bundle and allows more space within the interior of the handle body 12. The sleeve 62 further functions as a strain relief.

It is a particular advantage of the disclosed embodiments that a connector assembly is provided that facilitates ease of disconnection of the catheter sheath 14 from the handle body 12 of the catheter assembly 10. In addition, the electrical connector assembly 40, being substantially planar, is of a compact construction rendering the interior of the handle body 12 free of obstructions and allowing ancillary items, such as a deflection stylet (not shown) and the irrigation conduit 24, to easily pass through the interior of the handle body 12.

In addition, the ease of disconnection of the first connector member 42 from the second connector member 48 of the electrical connector assembly 40 readily lends the catheter assembly 10 for reprocessing purposes. Thus, costs involved with doing cardiac procedures using such catheters can be significantly reduced due to the ability to re-use the catheters after reprocessing. In this regard, the term “reprocessing” (and derivatives) is to be understood in a broad sense to include reprocessing, remanufacturing, refurbishment, or the like.

Reference throughout this specification to “one embodiment,” “some embodiments” or “an embodiment” means that a particular feature, structure or characteristic described in connection with the embodiment is included in at least one embodiment of the present invention. Thus, appearances of the phrases “in one embodiment,” “in some embodiments” or “in an embodiment” in various places throughout this specification are not necessarily all referring to the same embodiment, but may. Furthermore, the particular features, structures or characteristics in one or more embodiments may be combined in any suitable manner, as would be apparent to one of ordinarily skill in the art from this disclosure.

As used herein, unless otherwise specified, the use of ordinal adjectives “first,” “second,” “third,” etc., to describe a common object, merely indicate that different instances of like objects are referred to, and are not intended to imply that the objects so described must be in a given sequence, either temporally, spatially, in ranking, or in any other manner.

In the claims below and the description herein, any one of the terms “comprising,” “comprised of,” or “which comprises” is an open term that means including at least the elements/features that follow, but not excluding others. Thus, the term “comprising,” when used in the claims, should not be interpreted as being limitative to the means or elements or steps listed thereafter. For example, the scope of the expression “a device comprising A and B” should not be limited to devices consisting only of elements A and B. Any one of the terms “including,” “which includes,” or that “includes,” as used herein, is also an open term that also means including at least the elements/features that follow the term, but not excluding others. Thus, “including” is synonymous with and means “comprising.”

It should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

In the description provided herein, numerous specific details are set forth. However, it is understood that embodiments of the invention may be practiced without these specific details. In other instances, well-known methods, structures and techniques have not been shown in detail in order not to obscure an understanding of this description.

Similarly, it is to be noticed that the term “coupled” when used in the claims, should not be interpreted as being limited to direct connections only. The terms “coupled” and “connected,” along with their derivatives, may be used. It should be understood that these terms are not intended as synonyms for each other. Thus the scope of the expression “a device A coupled to a device B” should not be limited to devices or systems wherein an output of device A is directly connected to an input of device B. It means that there exists a path between an output of A and an input of B, which may be a path including other devices or means. “Coupled” may mean that two or more elements are either in direct physical or electrical contact, or that two or more elements are not in direct contact with each other but yet still co-operate or interact with each other.

Thus, while there has been described what are believed to be the preferred embodiments of the invention, those skilled in the art will recognize that other and further modifications may be made thereto without departing from the spirit of the invention, and it is intended to claim all such changes and modifications as falling within the scope of the invention. For example, any formulas given above are merely representative of procedures that may be used. Functionality may be added or deleted from the block diagrams and operations may be interchanged among functional blocks. Steps may be added or deleted to methods described within the scope of the invention.

It will be appreciated by persons skilled in the art that numerous variations and/or modifications may be made to the disclosure as shown in the specific embodiments without departing from the scope of the disclosure as broadly described. The present embodiments are, therefore, to be considered in all respects as illustrative and not restrictive.

1. A catheter electrical connector assembly, comprising:
a first connector member to which conductors of a catheter sheath are connectable; and
a second connector member to which conductors of an electrical connector of a catheter handle are connectable, the first connector member and the second connector member having complementary engaging formations for effecting disconnection between the first connector member and the second connector member, the first and second connector members further being of a compact construction to facilitate mounting in the catheter handle.

2. The assembly of claim 1, wherein the first connector member has a substantially planar configuration having a surface carrying conductive pads, the conductors of the catheter sheath being electrically connectable to the conductive pads.

3. The assembly of claim 2, wherein the second connector member comprises a receptacle defining a socket housing terminals, the first connector member being receivable in the socket with each conductive pad of the first connector member being associated with one of the terminals of the receptacle.

4. The assembly of claim 3, wherein the terminals of the receptacle are connectable via a cable arrangement to the electrical connector of the catheter handle.

5. The assembly of claim 4, wherein the second connector member comprises a printed circuit board on which the receptacle is mounted, the cable arrangement extending from the printed circuit board.

6. The assembly of claim 4, wherein the cable arrangement comprises a flexible printed circuit board on which the receptacle is mounted, the flexible printed circuit board being connectable to the electrical connector of the catheter handle.

7. A catheter electrical connector assembly comprising:
a first, card-shaped connector member to which conductors of a catheter sheath are connectable; and
a second connector member to which conductors of an electrical connector of a catheter handle are connectable, the second connector member defining a socket formation in which the first connector member is removably receivable.

8. A catheter assembly comprising:
a handle having a proximal end and a distal end and a passage extending between the proximal end and the distal end;
an electrical connector arranged at the proximal end of the handle with the electrical connector being in communication with the passage of the handle;
a catheter sheath extending from the distal end of the handle, the catheter sheath carrying at least one electrode at a distal region of the catheter sheath with conductors for the catheter sheath extending along the catheter sheath proximally to extend beyond a proximal end of the catheter sheath; and
an electrical connector assembly received in the passage of the handle, the electrical connector assembly including a first connector member electrically connected to the conductors of the catheter sheath, and a second connector member electrically connected to the electrical connector arranged at the proximal end of the handle, the first connector member and the second connector member having complementary engaging formations for effecting disconnection between the first connector member and the second connector member.

9. The catheter assembly of claim 8, wherein the catheter sheath defines at least one lumen and the conductors extend through the lumen.

10. The catheter assembly of claim 8, further comprising a protective element received over portions of the conductors extending beyond the proximal end of the catheter sheath.

11. The catheter assembly of claim 9, further comprising a protective element received over portions of the conductors extending beyond the proximal end of the catheter sheath.

12. The catheter assembly of claim 8, wherein the first connector member has a substantially planar configuration having a surface carrying conductive pads, the conductors of the catheter sheath being electrically connected to the conductive pads.

13. The catheter assembly of claim 12, wherein the second connector member comprises a receptacle defining a socket housing terminals, the first connector member being receivable in the socket with each conductive pad of the first connector member being associated with one of the terminals of the receptacle.

14. The catheter assembly of claim 13, wherein the terminals of the receptacle are connected by a cable arrangement to the electrical connector of the catheter handle.

15. The catheter assembly of claim 14, wherein the second connector member comprises a printed circuit board on which the receptacle is mounted, the cable arrangement extending from the printed circuit board.

16. The catheter assembly of claim 14, wherein the cable arrangement comprises a flexible printed circuit board on which the receptacle is mounted, the flexible printed circuit board being connected to the electrical connector of the catheter handle.

17. The catheter assembly of claim 8, wherein the first connector member is card-shaped, and wherein the second connector member defines a socket formation in which the first connector member is removably received.

18. A method of using a catheter assembly, comprising:
providing a catheter sheath carrying at least one electrode at a distal region of the catheter sheath with conductors extending along the catheter sheath proximally and extending beyond a proximal end of the catheter sheath, the catheter sheath further including a first connector member electrically connected to the conductors extending beyond the proximal end of the catheter sheath;
providing a handle having a proximal end, a distal end, and a passage extending between the proximal end and the distal end, the handle having an electrical connector at the proximal end of the handle, the handle further including a second connector member in the passage of the handle and electrically connected to the electrical connector at the proximal end of the handle;
attaching the first connector member of the catheter sheath to the second connector member of the handle to form an electrical connector assembly disposed in the passage of the handle;
mounting the catheter sheath to the distal end of the handle; and
using the catheter assembly to treat a patient.

19. The method of claim 18, further comprising:
removing the catheter sheath from the distal end of the handle and disconnecting the first connection member of the catheter sheath from the second connector member of the handle;
providing another catheter sheath;
attaching a first connector member of the another catheter sheath to the second connector member of the handle to form an electrical connector assembly disposed in the passage of the handle; and
mounting the another catheter sheath to the distal end of the handle.

20. The method of claim 19, further comprising using the handle and the another catheter sheath to treat a patient.
